(19) United States
(12) Patent Application Publication
(10) Pub. No.: US 2018/0075941 A1
(43) Pub. Date: Mar. 15, 2018

(54) LOW TEMPERATURE DIELECTRIC FLUID COMPOSITIONS

(71) Applicant: M&I Materials Limited, Manchester, Greater Manchester (GB)

(72) Inventors: Russell MARTIN, Manchester, Greater Manchester (GB); Francine Elizabeth EDWARDS, Manchester, Greater Manchester (GB)

(21) Appl. No.: 15/552,211

(22) PCT Filed: Feb. 22, 2016
(86) PCT No.: PCT/GB2016/050437

§ 371 Date: Aug. 18, 2017

(30) Foreign Application Priority Data
Feb. 20, 2015 (GB) 1502874.9

(51) Int. Cl. H01B 3/20; C07C 67/08

(52) U.S. Cl. CPC H01B 3/20; H01F 27/105; C07C 67/08

(57) ABSTRACT

The present invention relates to an ester composition that is suitable for successful operation in a dielectric fluid at extreme low temperatures, such as below about minus 50° C., without compromising the dielectric properties of the composition. The invention also relates to methods of manufacturing the ester composition and a dielectric fluid composition containing it.

The present invention relates to an ester composition that is suitable for use in a dielectric fluid at extreme low temperatures, without compromising the dielectric properties of the composition, as well as to methods of manufacturing the ester composition and a dielectric fluid composition containing it.

The use of dielectric fluids in electrical apparatus such as transformers is well known. Dielectric fluids are used throughout the world in transformers to provide electrical insulation, provide cooling and prevent excessive temperature rise within transformers, to suppress corona and arcing, and thus to prolong the lifetime of the transformer. Dielectric fluids known for such use include mineral oil based fluids, natural ester based fluids and synthetic ester based fluids. Known synthetic esters include those produced from the reaction of an alcohol with one or more carboxylic acids. Dielectric fluids based on such synthetic esters have a number of advantages over mineral oil based fluids, but there remains a need for synthetic esters having improved properties.

Particularly, the use of conventional dielectric fluids, as used in many thousands of transformers worldwide, has hitherto never been able to extend to extreme conditions. One such extreme condition is extremely low temperature, such as below about −50° C. At such low temperatures, the available dielectric fluid compositions are either in a solid form, or are so viscous that normal fluid flow is inhibited, and the compositions are unable to perform their desired functions.

There is therefore a need and a desire for an ester composition that is able to perform effectively as a dielectric fluid at extreme low temperatures, avoiding the problems experienced with existing compositions. Therefore, in accordance with a first aspect of the invention, there is provided an ester composition, wherein the ester composition comprises a plurality of esters derived from a reaction of:
i) one or more polyols, wherein the one or more polyols are each independently a straight chain or branched C₂-C₈ polyol; and
ii) two or more carboxylic acids, wherein the carboxylic acids are each independently a straight chain or branched C₄-C₁₂ carboxylic acid.

According to one embodiment of the invention, each of the one or more polyols may be a C₂, C₃, C₄, C₅, C₆, C₇, or C₈ polyol. Typically, each of the one or more polyols is selected from straight or branched C₂ to C₅ polyols, and may have a C₂ to C₃ backbone, with or without one or more hydrocarbon side groups. Where any of the polyols are branched, they typically have one or more C₁ or C₂ side groups, typically C₁.

By way of non-limiting examples, the polyol may be selected from neopentyl glycol (NPG), glycerol, butane diol, ethylene glycol and propylene glycol. More typically, only one polyol is used; the polyol is typically NPG.

According to another embodiment of the invention, the two or more carboxylic acids are typically each independently selected from straight chain or branched C₄, C₅, C₆, C₇, C₈, C₉, C₁₀, C₁₁ and C₁₂ carboxylic acids. More typically, the two or more carboxylic acids are typically each independently selected only from straight chain or branched C₇, C₈, C₉, C₁₀, C₁₁ and C₁₂ carboxylic acids and do not include any acids outside of this range. Still more typically, they are each independently selected only from straight chain or branched C₇, C₈, C₉, C₁₀ carboxylic acids, and do not include any acids outside of this range.

According to one embodiment of the invention, the polyol reacts with two or more carboxylic acids, more typically with only two carboxylic acids.

In this embodiment, typically, a first carboxylic acid is a C₇, C₈, or C₉ acid. More typically, it may be a C₈ acid, more typically a branched C₈ acid, such as, for example, 2-ethylhexanoic acid (2EHA).

Typically, the second carboxylic acid is a straight or branched C₈, C₉, or C₁₀ acid, more typically a straight chain C₈, C₉, or C₁₀ acid, i.e. n-octanoic acid, n-nonanoic acid, or n-decanoic acid. More typically, the acid is n-nonanoic acid.

Typically, the ester composition comprises esters formed from the reactions of a polyol with (i) a branched C₈ carboxylic acid as the first carboxylic acid; and (ii) a linear C₉ carboxylic acid as the second carboxylic acid.

According to one embodiment of the invention, the polyol is neopentylglycol (NPG), the first carboxylic acid is 2EHA, and the second carboxylic acid is n-nonanoic acid.

The resulting product from this reaction of one or more polyols and two carboxylic acids is not a pure substance and comprises a mixture of a number of possible ester structures. This ester mixture arises as a natural consequence of the reaction process. For example, NPG contains two alcohol functional groups, so the reaction of NPG with two acids (such as 2EHA, and a C₉ acid) would result in three different di-ester structures, the di-esters containing the functional groups of:

### 2EHA and 2EHA

### 2EHA and C₉

C₉ and C₉.

The relative quantities of the 2EHA and C₉ are typically in the region of 60-95 wt % 2EHA and 5-40 wt % C₉ acid, more typically about 70-90 wt % 2EHA and 10-30 wt % C₉ acid, still more typically about 75-85 wt % 2EHA and about 15-25 wt % C₉ acid.

According to a second embodiment of the invention, the polyol reacts with three or more carboxylic acids, and typically three carboxylic acids are used.

In this second embodiment, typically, a first carboxylic acid is a C₇, C₈, or C₉ acid. More typically, it may be a C₈ acid, more typically a branched C₈ acid, such as, for example, 2-ethylhexanoic acid (2EHA).

Typically, a second carboxylic acid is a C₈, C₉, or C₁₀ acid, such as, for example, n-octanoic acid, n-decanoic acid, or isononanoic acid (3,5,5-trimethylhexanoic acid). There may also typically be a third carboxylic acid, which may also be a C₈, C₉, or C₁₀ acid, such as, for example, n-octanoic acid, n-decanoic acid, or isononanoic acid, and which is different to the second carboxylic acid.

Typically, the ester composition comprises esters formed from the reactions of a polyol with (i) a branched C₈ carboxylic acid as the first carboxylic acid; (ii) a linear C₈ carboxylic acid and a linear C₁₀ carboxylic acid as the second and third carboxylic acids.

The relative quantities of the C₈ and C₁₀ carboxylic acids within any mixture of these carboxylic acids, prior to them being combined with the first carboxylic acid, is typically in the region of 50-70 wt % C₈ and 30-50 wt % C₁₀, more typically about 55-65 wt % C₈ and 35-45 wt % C₁₀, still more typically about 60 wt % C₈ and about 40 wt % C₁₀. Fₒᵣ the reaction with the one or more polyols, after the second and third carboxylic acids have been combined with the first carboxylic acid, there is typically in the region of about 10-15% of the second carboxylic acid on a molar basis, about 5-10% of the third carboxylic acid on a molar basis, with the remainder being the first carboxylic acid.

According to one embodiment of the invention, the polyol is neopentaglycol (NPG), the first carboxylic acid is 2EHA, and the second and third carboxylic acids are a mixture of different C₈, C₉, or C₁₀ carboxylic acids. Typically, the reaction acid mixture has a mole ratio of about 60-95% 2EHA to about 5-40% C₈-C₁₀ carboxylic acids, more typically 70-90% 2EHA to about 10-30% C₈-C₁₀ carboxylic acids, more typically about 80% 2EHA to about 20% C₈-C₁₀ carboxylic acids. Typically, the second and third carboxylic acids are a mixture of n-octanoic acid (C₈) and decanoic acid (C₁₀).

The resulting product from this reaction of one or more polyols and three carboxylic acids is not a pure substance and comprises a mixture of a number of possible ester structures. This ester mixture arises as a natural consequence of the reaction process. For example, NPG contains two alcohol functional groups, so the reaction of NPG with three acids (such as 2EHA, C₈ acid and a C₁₀ acid) would result in six different di-ester structures, the di-esters containing the functional groups of:

### 2EHA and 2EHA

### 2EHA and C8

### 2EHA and C10

### C8 and C8

### C8 and C10

### C10 and C10.

The ester composition may comprise small amounts of unreacted alcohol and/or acids as impurities. Typically, the ester composition is substantially free of alcohol and/or acids.

The ester composition of the invention has dielectric properties, and is suitable for use as a dielectric fluid, particularly for use as a dielectric fluid at extremely low temperatures, such as below about minus 50° C., below about minus 60° C., below about minus 70° C., and even down to about minus 75° C.

In terms of the properties of the synthetic ester composition of the invention, it has been observed that it has a much lower pour point than an existing leading commercial dielectric fluid such as Midel 7131 (minus 75° C. as compared to minus 60° C.) and has a viscosity that is approximately equal to mineral oil at 40° C. Consequently, the ester composition of the invention may be employed in a dielectric fluid without the need for a pour point depressant. However, if desired, a pour point depressant may be used.

The synthetic ester composition of the invention also has a fire point of around 220° C., and so is more fire safe than mineral oil, which has a fire point of around 170° C. Crucially, these advantages do not compromise the dielectric properties of the ester composition of the invention. The ester composition of the invention has a breakdown voltage comparable with Midel 7131, and is readily biodegradable and oxygen stable.

Also envisaged within the present invention is that the ester composition of the invention may be used as a low temperature lubricant composition.

Typically, the ester composition has a pour point of minus 50° C. or less when measured according to the method of ISO 3016, more typically minus 55° C. or less, more typically −minus 60° C. or less, more typically minus 65° C. or less, or even minus 70° C. or less. Typically, the pour point is about minus 75° C., or even less.

Typically, the ester composition has a viscosity of 20 cP or less at 40° C. measured using a Brookfield DV-I Prime Viscometer; more typically of 15 cP or less at 40° C., or of 10 cP or less at 40° C., or of 3-10 cP or less at 40° C. Typically, said viscosity comprises dynamic viscosity.

Typically, the ester composition has a COC Fire point of 200° C. or higher measured according to the method of ISO 2592; more typically 210° C. or higher, or 220° C. or higher.

According to a further aspect of the invention, there is provided a dielectric fluid composition comprising:

(I) an ester composition, wherein the ester composition comprises a plurality of esters derived from a reaction of:
i) one or more polyols, wherein the one or more polyols are each independently a straight chain or branched C₂-C₈ polyol; and
ii) two or more carboxylic acids, wherein each of the carboxylic acids are each independently a straight chain or branched C₄-C₁₂ carboxylic acid; and

(II) one or more additives.

The additives are typically selected from antioxidants, metal deactivators and pour point depressants, and combinations thereof.

Typically, the dielectric fluid composition comprises the ester composition (I) in an amount of at least 95% by weight of the dielectric fluid composition. Suitably, the dielectric fluid composition comprises the ester composition (I) in an amount of at least 96% by weight of the composition, for example in an amount of at least: 97%, 98% or 99% by weight of the composition. Typically, the dielectric fluid composition comprises the ester composition (I) in an amount of at least 99.5% by weight of the composition.

Typically, the dielectric fluid composition comprises the additives (II) in the following amounts:
one or more antioxidants in a total amount of about 0.0001% to about 1% by weight of the composition; and/or
one or more metal deactivators in a total amount of about 0.0001% to about 1% by weight of the composition; and/or
one or more pour point depressants in a total amount of 0% to about 1% by weight of the composition.

Combinations of any two or more of these additives may be used, as desired.

Typically, the dielectric fluid composition comprises an antioxidant in an amount of at least about 0.0001% by weight of the composition, more typically in an amount of at least about 0.001%, at least about 0.01%, at least about 0.1%, at least about 0.25% by weight of the composition, for example in an amount of about 0.25% by weight of the composition.

The antioxidant may comprise a phenolic antioxidant, such as a sterically hindered phenolic antioxidant.

Typically, the dielectric fluid composition comprises a metal deactivator in an amount of at least about 0.0001% by weight of the composition; more typically in an amount of at least about 0.001%, at least about 0.002%, or at least about 0.005% by weight of the composition, for example in an amount of about 0.005% by weight of the composition.

Suitably, the dielectric fluid composition comprises one or more additives (II) selected from antioxidants and metal deactivators.

The dielectric fluid composition may be substantially or completely free from pour point depressant. Alternatively, the dielectric fluid composition may comprise a pour point depressant. Typically, the ester composition (I) is suitable for use as a dielectric fluid without the need to be combined with a pour point depressant.

Typically, the dielectric fluid composition comprises an ester composition (I) and additives (II) in a combined amount of at least about 95% by weight of the composition, typically at least about 99% by weight of the composition, more typically in a combined amount of at least about 99.9% by weight of the composition. Typically, the dielectric fluid composition consists of an ester composition (I) and additives (II).

Typically, the dielectric fluid composition comprises an ester composition, an antioxidant and a metal deactivator in a combined amount of at least 95% by weight of the composition, more typically in a combined amount of at least 99% by weight of the composition, more typically in a combined amount of at least 99.9% by weight of the composition. Typically, the dielectric fluid composition consists of an ester composition, an antioxidant and a metal deactivator.

The dielectric fluid composition may comprise minor or trace amounts of unreacted alcohol and/or acids as impurities. Suitably, the dielectric fluid composition is substantially free of alcohol and/or acids.

Typically, the dielectric fluid composition has a pour point of less than minus 50° C., when said pour point is measured according to the method of ISO 3016.

Typically, the dielectric fluid composition has a pour point of minus 55° C. or less.

Typically, the dielectric fluid composition has a pour point of minus 60° C. or less.

Typically, the dielectric fluid composition has a pour point of minus 65° C. or less.

Typically, the dielectric fluid composition has a pour point of minus 70° C. or less.

Typically, the dielectric fluid composition has a pour point of minus 75° C. or less.

Typically, the dielectric fluid composition has a viscosity of 20 cP or less at 40° C. measured using a Brookfield DV-I Prime Viscometer; more typically of 15 cP or less at 40° C., or of 10 cP or less at 40° C., or of 3-10 cP or less at 40° C. Typically, said viscosity comprises dynamic viscosity.

Typically, the dielectric fluid composition has a COC Fire point of 200° C. or higher measured according to the method of ISO 2592; more typically 210° C. or higher, or 220° C. or higher.

According to another aspect of the present invention there is provided a method of manufacturing an ester composition, wherein the method comprises forming a plurality of esters by reacting:
i) one or more polyols, wherein the one or more polyols are each independently a straight chain or branched C₂-C₈ polyol; and
ii) two or more carboxylic acids, wherein each of the carboxylic acids are each independently a straight chain or branched C₄-C₁₂ carboxylic acid.

The polyols and carboxylic acids for use in the reaction are as defined hereinabove.

Typically, the method comprises reacting the polyol with the carboxylic acids wherein the acids are in excess by an amount of at least 10 molar %; more typically in an excess of at least 20 molar %, for example an excess of 30 molar %.

Typically, the method comprises removing water as it is formed. Any excess acid may be removed following the reflux stage. If necessary, the reaction mixture may be adjusted to neutral pH—i.e. between about 6-8—following the reflux stage. Typically, the method comprises treating the ester composition.

Typically, the method comprises adding alumina and/or subjecting the reaction mixture to a purifying powder treatment and/or adding an antioxidant. The ester composition may be filtered during the method. The antioxidant may be added with heating, typically prior to filtering. According to another embodiment of the present invention, there is provided a method of manufacturing a dielectric fluid composition comprising an ester composition, wherein the method comprises combining an ester composition with an additive, wherein the ester composition comprises a plurality of esters derived from the reaction of:
i) one or more polyols, wherein the one or more polyols are each independently a straight chain or branched C₂-C₈ polyol; and
ii) two or more carboxylic acids, wherein each of the carboxylic acids are each independently a straight chain or branched C₄-C₁₂ carboxylic acid.

The polyols and carboxylic acids for use in the reaction are as defined hereinabove.

The additive may be selected from antioxidants, metal deactivators and pour point depressants, and combinations thereof. The various additives and their respective amounts are also defined hereinabove.

Typically, the method comprises adding an antioxidant, which may be added with or without heating. The antioxidant may also be added prior to, or after, any filtering of the ester composition.

If a metal deactivator is added as an additive, the metal deactivator may be added prior to, or after, any filtering of the ester composition.

The dielectric fluid may be used in an electrical apparatus. The electrical apparatus may be a transformer, such as a high voltage transformer. Also provided within the present invention is an electrical apparatus containing the dielectric fluid composition defined herein.

According to another embodiment of the present invention, there is provided the use of an ester composition as defined hereinabove in or as a dielectric fluid.

The present invention will now be illustrated by way of the following examples, which are intended to be exemplary only, and in no way limiting upon the scope of the invention.

### EXAMPLE 1

Neopentyl glycol (265.6 g), 2-Ethylhexanoic acid (748 g), and an octanoic/decanoic acid blend (201.3 g, approximately 60% octanoic/40% decanoic acid) were added to a 2-Litre round bottom flask fitted with dean-stark apparatus and a condenser. The reaction mixture was stirred at 80° C. for one hour in the presence of alumina to neutralise the reaction mixture, subjected to a purifying powder treatment, and an antioxidant were added. The ester was filtered twice, a metal deactivator was added, and the ester was degassed until the moisture content of the ester was less than 80 ppm.

### EXAMPLE 2

Neopentyl glycol (258.8 g), 2-Ethylhexanoic acid (745.5 g), and n-nonanoic acid blend (204.5 g) were added to a 2-Litre round bottom flask fitted with dean-stark apparatus and a condenser. The reaction mixture was stirred at 80° C. for one hour in the presence of alumina to neutralise the reaction mixture, subjected to a purifying powder treatment, and an antioxidant was added. The ester was filtered twice, a metal deactivator was added, and the ester was degassed until the moisture content of the ester was less than 80 ppm.

The properties of the ester compositions of the invention are shown in Table 1 below, together with the comparative data of the Midel 7131 commercial product.

[NOTE: I've added the example for the C9 prep. If there is anything more you would like to see taken out of the example information, let me know]

TABLE 1

| | | | | Midel | |
|---|---|---|---|---|---|
| Property | Units | Example 1 | Example 2 | 7131 | Test Method |
| Water content | ppm | 50 | 50 | 50 | IEC 60814 |
| Acid Value | mgKOH/g | <0.03 | <0.03 | <0.03 | IEC 62021-2 |
| Colour | HU | 100 | 100 | 125 | ISO 2211 |
| Tan delta at 90° C. and | | <0.03 | <0.03 | <0.008 | IEC 60247 |
| 50 Hz | | | | | |
| Volume resistivity DC | GΩcm | >10 | >10 | >30 | IEC 60247 |
| at 90° C. | | | | | |
| Breakdown voltage | kV | >75 | >75 | >75 | IEC 60156 |
| Viscosity at 100° C. | cP | 1.86 | | 4.79 | ISO 3104 |
| Viscosity at 40° C. | cP | 6.92 | | 26.8 | |
| Viscosity at 0° C. | cP | 38.6 | 42.1 | 236 | |
| Viscosity at −10° C. | cP | 84.4 | 79.4 | 427 | |
| Viscosity at −20° C. | cP | 167 | 172 | 1400 | |
| Viscosity at −30° C. | cP | 391 | 434 | 4229 | |
| Viscosity at −40° C. | mm² s⁻¹ | | 1330 | 16090 | |
| Viscosity at −50° C. | mm² s⁻¹ | | 5060 | 89160 | |
| Density at 20° C. | kg dm⁻³ | 0.92 | 0.92 | 0.97 | ISO 3675 |
| PMCC flash point | ° C. | 190 | 190 | 260 | ISO 2719 |
| COC fire point | ° C. | 216 | 220 | 316 | ISO 2592 |
| Pour point | ° C. | −75 | −72 | −60 | ISO 3016 |
| | | | | | (modified)/ISO |
| | | | | | 3016 |

[NOTE: I've added the C9 data into a new column in this table, though as some of the numbers for MIdel 7131 that Francine provided earlier weren't quite the same as in the table we filed, I've left those numbers alone]

As can be seen from the above, the dielectric fluid composition of Example 1 has physical and electrical properties which render it particularly suitable for use and successful operation as a dielectric fluid in electrical apparatuses in extreme low temperatures, in contrast to the commercially available dielectric fluids which are not intended for use in such extreme conditions. In particular, the pour point is 15° C. lower for the composition of the invention, and the viscosity is significantly lower at the various temperatures at which it is measured.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

It is of course to be understood that the present invention is not intended to be restricted to the foregoing specific embodiments, which are described by way of example only. The invention extends to any novel feature, or combination of features, disclosed in this specification (including any accompanying claims, abstract and drawings), or to any novel one, or any novel combination, of the steps of any method or process so disclosed.

1. An ester composition, wherein the ester composition comprises a plurality of esters derived from a reaction of:
i) one or more polyols, wherein the one or more polyols are each independently a straight chain or branched C₂-C₈ polyol; and
ii) two or more carboxylic acids, wherein the carboxylic acids are each independently a straight chain or branched C₄-C₁₂ carboxylic acid.

2. An ester composition according to claim 1, wherein the one or more polyols are each independently selected from straight or branched C₂ to C₅ polyols.

3. An ester composition according to claim 2, wherein the one or more polyols each independently have a C₂ to C₃ backbone and one or more C₁ or C₂ hydrocarbon side groups.

4. An ester composition according to any preceding claim, wherein the one or more polyols each independently comprise neopentyl glycol (NPG), glycerol, butane diol, ethylene glycol or propylene glycol.

5. An ester composition according to any preceding claim, wherein only one polyol is used.

6. An ester composition according to claim 5, wherein the polyol comprises neopentyl glycol.

7. An ester composition according to any preceding claim, wherein the one or more polyols each react only with first and second carboxylic acids.

8. An ester composition according to claim 7, wherein the first carboxylic acid comprises a C₇, C₈, or C₉ acid.

9. An ester composition according to claim 8, wherein the first carboxylic acid comprises a branched C₈ acid.

10. An ester composition according to claim 8 or claim 9, wherein the first carboxylic acid comprises 2-ethylhexanoic acid.

11. An ester composition according to any of claims 7-10, wherein the second carboxylic acid is selected from a straight chain C₈, C₉, or C₁₀ acid.

12. An ester composition according to any of claims 7-11, wherein the second carboxylic acid is n-nonanoic acid.

13. An ester composition according to any preceding claim, wherein the one or more polyols comprise neopentylglycol, and the one or more polyols react only with first and second carboxylic acids, wherein the first carboxylic acid is 2-ethylhexanoic acid, and the second carboxylic acid is n-nonanoic acid.

14. An ester composition according to claim 13, wherein the one or more polyols comprise about 60-95 wt % 2-ethylhexanoic acid and about 5-40 wt % n-nonanoic acid.

15. An ester composition according to any preceding claim, wherein the one or more polyols each react with first, second and third carboxylic acids.

16. An ester composition according to claim 15, wherein the first carboxylic acid comprises a C₇, C₈, or C₉ acid.

17. An ester composition according to claim 16, wherein the first carboxylic acid comprises a branched C₈ acid.

18. An ester composition according to claim 16 or claim 17, wherein the first carboxylic acid comprises 2-ethylhexanoic acid.

19. An ester composition according to any of claims 16-18, wherein the second and third carboxylic acids are different to each other and are each independently selected from a straight chain or branched C₈, C₉, or C₁₀ acid.

20. An ester composition according to claim 19, wherein the second and third carboxylic acids are different to each other and are each independently selected from n-octanoic acid, n-decanoic acid, or isononanoic acid (3,5,5-trimethylhexanoic acid).

21. An ester composition according to any preceding claim, wherein the one or more polyols comprise neopentaglycol, and the one or more polyols react with first, second and third carboxylic acids, wherein the first carboxylic acid is 2-ethylhexanoic acid, and the second and third carboxylic acids comprise a mixture of two different straight chain or branched C₈, C₉, or C₁₀ carboxylic acids.

22. An ester composition according to claim 21, wherein the second and third carboxylic acids comprise a mixture of n-octanoic acid and decanoic acid.

23. An ester composition according to claim 22, wherein the second and third carboxylic acids comprise a mixture of about 50-70 wt % n-octanoic acid and about 30-50 wt % decanoic acid.

24. An ester composition according to any of claims 16-23, wherein the mixture of the first, second and third carboxylic acids comprises between about 70 to about 90 wt % of the first carboxylic acid and between about 10 to about 30 wt % of a combination of the second and third carboxylic acids.

25. A dielectric fluid composition comprising:
(I) an ester composition according to any of claims 1-24; and
(II) one or more additives.

26. A dielectric fluid composition according to claim 25, wherein the additives are selected from antioxidants, metal deactivators and pour point depressants, and combinations thereof.

27. A dielectric fluid composition according to claim 25, wherein the additives do not include a pour point depressant.

28. A dielectric fluid composition according to any of claims 25-27, wherein the dielectric fluid composition comprises the ester composition (I) and additives (II) in a combined amount of at least 99% by weight of the composition.

29. A dielectric fluid composition according to any of claims 26-28, wherein the dielectric fluid composition comprises an antioxidant in an amount of at least 0.0001% by weight of the composition and/or a metal deactivator in an amount of at least 0.0001% by weight of the composition.

30. An electrical apparatus containing a dielectric fluid composition according to any of claims 25-29.

31. A method of manufacturing an ester composition according to any of claims 1-24, wherein the method comprises forming a plurality of esters by reacting:
(i) one or more polyols, wherein the one or more polyols are each independently a straight chain or branched C₂-C₈ polyol; and
(ii) two or more carboxylic acids, wherein each of the carboxylic acids are independently a straight chain or branched C₄-C₁₂ carboxylic acid.

32. A method of manufacturing a dielectric fluid composition comprising an ester composition according to any of claims 1-24, wherein the method comprises combining the ester composition with an additive, wherein the ester composition comprises a plurality of esters derived from the reaction of:
(i) one or more polyols, wherein the one or more polyols are each independently a straight chain or branched C₂-C₈ polyol; and
(ii) two or more carboxylic acids, wherein each of the carboxylic acids are independently a straight chain or branched C₄-C₁₂ carboxylic acid.

33. A method according to claim 32, wherein the additives are selected from antioxidants, metal deactivators and pour point depressants, and combinations thereof.

34. A method according to claim 33, wherein the additives do not include a pour point depressant.

35. The use of an ester composition according to any of claims 1-24 and/or an ester composition manufactured according to the method of claim 25 as a dielectric fluid.

36. An ester composition, dielectric fluid composition, method of manufacturing an ester composition, method of manufacturing a dielectric fluid composition, or use of an ester composition substantially as herein described.
